(19) United States
(12) Patent Application Publication
(10) Pub. No.: US 2019/0308987 A1
(43) Pub. Date: Oct. 10, 2019

(54) DUAL MODULATORS OF BOTH MU AND KAPPA OPIOID RECEPTORS

(71) Applicant: VIRGINIA COMMONWEALTH UNIVERSITY, Richmond, VA (US)

(72) Inventors: Yan ZHANG, Glen Allen, VA (US); Dana SELLEY, Richmond, VA (US); William DEWEY, Richmond, VA (US); Hamid AKBARALI, Richmond, VA (US)

(21) Appl. No.: 16/306,232

(22) PCT Filed: May 26, 2017
(86) PCT No.: PCT/US17/34641

§ 371 Date: Nov. 30, 2018

Related U.S. Application Data
(60) Provisional application No. 62/344,618, filed on Jun. 2, 2016.

(51) Int. Cl. C07D 489/08; A61P 1/00

(52) U.S. Cl. CPC C07D 489/08; A61P 1/00

(57) ABSTRACT

Peripherally selective compounds that modulate both the mu opioid receptor (MOR) and the kappa opioid receptor (KOR) are provided. The compounds are substituted derivatives of 6p-N-heterocyclic naltrexamine (NAP) and are used in the treatment of diseases involving visceral pain such as irritable bowel syndrome (IBS), opioid induced constipation (OIC), and others.

### CROSS-REFERENCE TO RELATED APPLICATIONS

This application claims benefit of U.S. provisional patent application 62/344,618, filed Jun. 2, 2016, the complete contents of which is hereby incorporated by reference.

### STATEMENT OF FEDERALLY SPONSORED RESEARCH AND DEVELOPMENT

This invention was made with government support under grant numbers DA024022, DA024009 and DA007027 awarded by the National Institutes of Health/National Institute on Drug Abuse. The United States government has certain rights in the invention.

### BACKGROUND OF THE INVENTION

### Field of the Invention

The invention generally relates to peripherally selective compounds that act as dual modulators, modulating both the mu opioid receptor (MOR) and the kappa opioid receptor (KOR). In particular, the invention provides substituted derivatives of 6β-N-heterocyclic naltrexamine (NAP) which have improved peripheral selectivity for both MOR and KOR, for use in the treatment of diseases involving visceral pain such as irritable bowel syndrome (IBS), opioid induced constipation (OIC), and others.

### Background

Opioids are the most commonly prescribed medications for the treatment of malignant and non-malignant pain. Despite their proven analgesic efficacy, significant side effects such as addiction, nausea, dizziness, urinary retention, and constipation limit the clinical utility of these drugs, particularly with chronic use.¹ Among these side effects, opioid-induced constipation (OIC) is one of the most common and distressing. It is estimated that up to 40% of patients on opioid treatment experience OIC.² OIC is often so debilitating that patients forego opioid treatment and suffer with their pain. The analgesic effects of opioids are primarily facilitated through activation of mu opioid receptors (MORs) located on neurons within the central and peripheral nervous system. Within the enteric nervous system, activation of MORs mediate the constipating effects of opioids.³ Propulsion within the gastrointestinal (GI) tract is the consequence of the concerted actions of circular muscles and longitudinal muscles that grind and propel the food bolus forward. Each of these actions is controlled by neurons of the myenteric plexus. Inhibition of neurotransmitter release is a primary mechanism by which MORs inhibit peristalsis.

Treatment of OIC is a significant challenge as laxative therapy is often ineffective.⁴ Peripheral MOR antagonists have been recently developed that can prevent or reverse OIC.⁵ Methylnaltrexone (MNTX, 2, FIG. 1) and Alvimopan (3, FIG. 1) are two peripherally selective MOR antagonists approved by the FDA for the treatment of OIC.⁶ However, MNTX suffers from low activity at producing spontaneous bowel movements and prolonged use of Alvimopan increases the risk of myocardial infarction.⁷ Therefore, the development of peripherally selective MOR antagonists would be of great benefit to patients suffering from OIC, as well as other gastrointestinal neuropathies such as irritable bowel syndrome (IBS).

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Described herein are derivatives of the 6β-N-heterocyclic substituted naltrexamine derivative NAP (4, FIG. 1) containing structural modifications, including selective alkylation of the pyridal nitrogen. NAP was previously identified as a novel MOR antagonist with peripheral selectivity and a 300 fold greater potency than methylnaltrexone. Further studies in the pharmacology of NAP demonstrated that it has mixed partial agonist and antagonist activity, with a bias towards antagonism of the β-arrestin 2 pathway. The NAP derivatives described herein display limited penetration into the central nervous system and thus exhibit improved peripheral selectivity. In addition, the compounds act as dual modulators, modulating both the mu opioid receptor (MOR) and the kappa opioid receptor (KOR). The disclosed compounds are thus useful for the treatment of disorders such as irritable bowel syndrome, e.g. patients suffering from visceral pain that is accompanied by one or both of constipation and diarrhea, and opioid induced constipation (OIC).

It is an object of this invention to provide compounds of Formula I

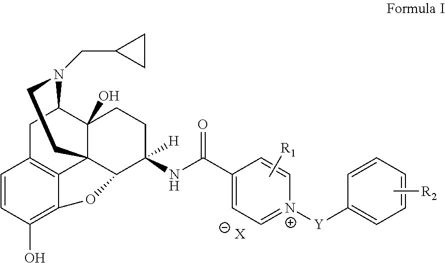

where

X is an anion; R1 and R2 are independently H, a straight chain or branched alkyl group, a straight chain or branched alkene group, an electron withdrawing group, or an electron donating group and may be present or absent; and Y is a linking or spacer group.

In some aspects, X is selected from the group consisting of fluoride, chloride, bromide, acetate, formate, bromate, pyruvate, nitrate, isocitrate, cis-aconitate, trans-aconitate, selenium oxoanion, maleate, malonate, phosphate, citrate, sulfate, oxalate, uric acid and choline. In some aspects, R1 is methyl, ethyl, fluoro, nitro or methoxyl. In additional aspects, R2 is methyl, ethyl, fluoro, nitro or methoxyl. In some aspects, Y is an atom or molecule capable of forming at least two covalent bonds, a first of which is with a nitrogen moiety of Formula I and a second of which is with a phenyl moiety of Formula I. In certain aspects, Y is selected form the group consisting of CH₂, C₂H₄, COCH₂, CONH, CH₂COCH₂, CH₂CONH, CH₂COOCH₂ and CH₂CONHCH₂. In further aspects, the compound is as shown in Formula II:

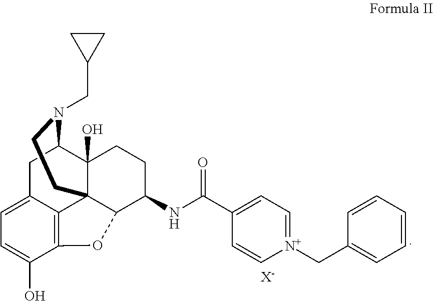

The invention also provides method for treating opioid induced constipation in a subject in need thereof, comprising administering to the subject a therapeutically effective amount of a compound of Formula I

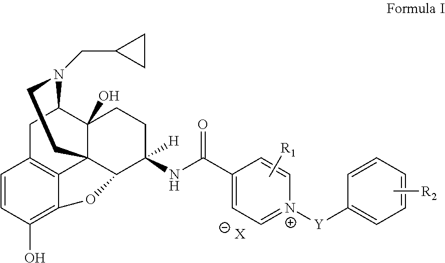

where

X is an anion; R1 and R2 are independently H, a straight chain or branched alkyl group, a straight chain or branched alkene group, an electron withdrawing group, or an electron donating group and may be present or absent; and Y is a linking or spacer group. In some aspects the compound is as shown in Formula II:

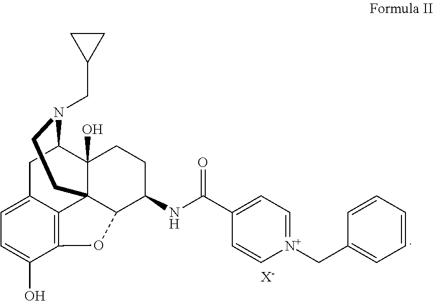

In yet further aspects, the invention provides methods for treating irritable bowel syndrome (IBS) in a subject in need thereof, comprising administering to the subject a therapeutically effective amount of a compound of Formula I

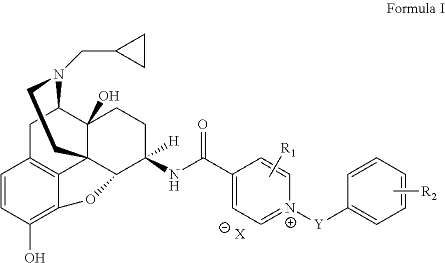

where

X is an anion; R1 and R2 are independently H, a straight chain or branched alkyl group, a straight chain or branched alkene group, an electron withdrawing group, or an electron donating group and may be present or absent; and Y is a linking or spacer group. In some aspects, the compound is as shown in Formula II:

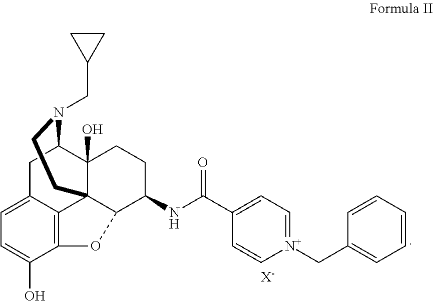

### BRIEF DESCRIPTION OF THE DRAWINGS

FIG. 1. Morphine and three known peripherally selective mu-opioid receptor antagonists.

FIG. 2A-C. Ligand docking study of BNAP (orange balls and sticks) in the MOR (A) and KOR (B) and DOR (C) crystal structures.

FIG. 3. BNAP effects on morphine antinociception when administered via subcutaneous (s.c) injection. Data point are mean responses±S.E.M, n=5.

FIG. 4. BNAP effects on morphine antinociception when administered via intracerebroventricular (ICV) injection. Data point are mean responses±S.E.M, n=5.

FIG. 5A-C. (A) Raw traces of morphine induced contractions in the mouse distal and proximal colon from the same animal. (B) Graph indicating the concentration dependent stimulation of contractions in response to morphine in the distal and proximal colon. (C) Histogram depicting the >5 fold difference in sensitivity between the distal and proximal colon. (Data points are mean responses±S.E.M of no less than three independent runs).

FIG. 6A-D. Morphine dose response curves in the presence of NAP or BNAP in distal (A, B) and proximal (C, D) colon. Data point are mean responses±S.E.M, n=4-6.

FIGS. 7A and B. (A) Effect of Morphine and BNAP on gastrointestinal transit as measured by bead expulsion. (B) Effect of Morphine and BNAP on gastrointestinal transit as measured by charcoal gavage. Data point are mean responses±S.E.M, n=6-8.

FIG. 8 A-C. The inhibition of electrical field stimulated contractions by opioid agonist. (A) Inhibition of EFS by the MOR full agonist DAMGO and the newly prepared KOR partial agonist BNAP. (B) Attempts at blocking the effects of DAMGO with the selective KOR antagonist nor-BNI. No significant change was observed. (C) Blocking of BNAP effects by nor-BNI. The concentration response curve is shifted to the right suggesting antagonism of BNAP by nor-BNI. Data point are mean responses±S.E.M, n=4-6.

FIG. 9A-C. (A) Analgesic activity of BNAP in the acetic acid induced writhing assay. Data point are mean responses±S.E.M. (B) Analgesic activity of BNAP in the acetic acid induced writhing assay in mice chronically treated with a 75 mg morphine pellet (MP) or placebo pellet (PP). (C) Prevention of BNAP analgesia in the presence of 10 mg/kg nor-BNI.

### DETAILED DESCRIPTION

Described herein are derivatives of NAP containing structural modifications, including selective alkylation of the pyridal nitrogen of NAP. The resulting compounds display limited penetration into the central nervous system and are thus peripherally selective, compared to the parent compound NAP. The compounds are useful for the treatment of disorders such as irritable bowel syndrome, all types of visceral pain that are accompanied by one or both of constipation and diarrhea, and opioid induced constipation (OIC).

The compounds described herein have a general formula as shown in Formula I

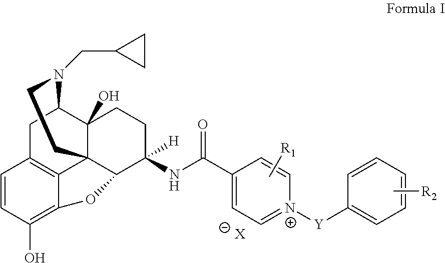

where

X is an anion,

R1 and R2 are independently H, a straight chain or branched alkyl group, a straight chain or branched alkene group, an electron withdrawing group, or an electron donating group and may be present or absent;

and

Y is a linking or spacer group comprising at least one an atom or molecule capable of forming at least two covalent bonds, a first of which is with a nitrogen moiety of Formula I and a second of which is with a phenyl moiety of Formula I.

Exemplary anions that may be associated with the compound include but are not limited to fluoride, chloride, bromide, acetate, formate, bromate, pyruvate, nitrate, isocitrate, cis-aconitate, trans-aconitate, selenium oxoanion, maleate, malonate, phosphate, citrate, sulfate, oxalate, uric acid, choline, etc.

Exemplary R1 and R2 substituents include various suitable straight or branched, saturated or unsaturated carbon chains, including but not limited to those that contain from about 1 to about 20 carbon atoms. In some aspects, the carbon chain is saturated, i.e. the carbon chain is an alkyl chain. Examples of suitable alkyl chains include but are not limited to methyl, ethyl, propyl, isopropyl, butyl, pentyl, heptyl, septyl, octyl, nonyl, decyl, undecyl and dodecyl, as well as branched isomers thereof, and substituted variants thereof. Alternatively, the branched or unbranched carbon chain may be unsaturated alkenes, e.g. containing one or more (e.g. 1, 2, 3, 4, or 5 or more) double bonds. For example, suitable lower alkenes containing 1 double bond include but are not limited to e.g. ethane, propene, butene, etc.

For both R1 and R2, suitable electron withdrawing groups include but are not limited to, e.g. halogens such as F, CN, NO₂, SO₃, CF₃, NR₂, OR, NHCOR (where R is H or a lower alkyl with from about 1-5 carbon atoms such as methyl, ethyl, propyl, butyl, pentyl, etc., or an alkene equivalent).

For both R1 and R2, suitable electron donating groups include but are not limited to, e.g. alkyl groups; alkoxyl groups (e.g. OR where R═H or a lower alkyl, e.g. methoxyl); alcohol groups (methanol, ethanol, propanol, etc.); amino and substituted amino groups, etc. Exemplary functional groups include but are not limited to OCH₃, phenyl and substituted phenyl.

Exemplary linker or spacer groups that may be present in the molecule. By “linker” or “spacer” we mean an atom or molecule capable of forming at least two covalent bonds, a first of which is with a nitrogen moiety of Formula I and a second of which is with a phenyl moiety of Formula I. Such linkers include but are not limited to saturated or unsaturated branched or unbranched carbon chains containing from about 1 to 20 carbon atoms, which may be substituted or unsubstituted. In some aspects, the carbon chain is saturated, i.e. the carbon chain is an alkyl chain. Examples of suitable alkyl groups include but are not limited to methyl, ethyl, propyl, isopropyl, butyl, pentyl, heptyl, septyl, octyl, nonyl, decyl, undecyl and dodecyl, as well as branched isomers thereof. Alternatively, the branched or unbranched carbon chain may be unsaturated alkenes, e.g. containing one or more (e.g. 1, 2, 3, 4, or 5 or more) double bonds. For example, suitable lower alkenes containing 1 double bond include but are not limited to e.g. ethene, propene, butene, etc. In addition, the carbon chains may be substituted, e.g. with atoms or atomic groups such as O, CO, N, NH₂, S, etc.

The linkers may include, for example, amides, esters, ethers, cyano, nitro, groups, etc. Other types of linker or spacer groups that may be present in the compounds include but are not limited to: carboxylates, thiolyl, etc.

In some aspects, the compound is as shown in Formula II:

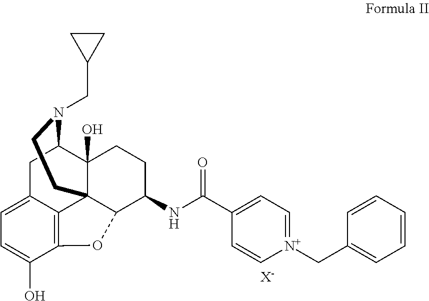

where X is an anion as described above for Formula I.

The compounds described herein generally have a binding affinity (Ki) for MOR in the range of from about 10.0 to about 0.05 nM, e.g. about 0.05, 0.1, 0.15, 0.2, 0.25, 0.3, 0.35, 0.4, 0.45, 0.5, 0.6, 0.7, 0.8, 0.9, 1.0, 2.0, 3.0, 4.0, 5.0, 6.0, 7.0, 8.0, 9.0 or about 10.0 including all intervening decimals to two decimal places. Generally, the compounds have a Ki for the KOR in the range of from about 50.0 to about 0.05 nM, e.g. about 5.0, 4.5, 4.0, 3.5, 3.0, 2.5, 2.0, 1.5, or 1.0 including all intervening integers and decimals to two decimal places. Further, the compounds generally have a Ki for the DOR that is greater than about 100 nM or more, e.g. about 100, 200, 300, 400, 500, 550, 600, 650, 700, 750, 800, 850, 900, 950 or 1000, including all intervening integers. The selectivity for MOR over DOR is typically at least about 100, and the selectivity for KOR over DOR is typically at least about 50.

The compounds described herein are generally delivered (administered) as a pharmaceutical composition. Such pharmaceutical compositions generally comprise at least one of the disclosed compounds, i.e. one or more than one (a plurality) of different compounds (e.g. 2 or more such as 2, 3, 4, 5, 6, 7, 8, 9, 10 or more) may be included in a single formulation. Accordingly, the present invention encompasses such formulations and compositions. The compositions generally include one or more substantially purified compounds as described herein, and a pharmacologically suitable (physiologically compatible) carrier, which may be aqueous or oil-based. In some aspects, such compositions are prepared as liquid solutions or suspensions, or as solid forms such as tablets, pills, powders and the like. Solid forms suitable for solution in, or suspension in, liquids prior to administration are also contemplated (e.g. lyophilized forms of the compounds), as are emulsified preparations. In some aspects, the liquid formulations are aqueous or oil-based suspensions or solutions. In some aspects, the active ingredients are mixed with excipients which are pharmaceutically acceptable and compatible with the active ingredients. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, pharmaceutically acceptable salts and the like. In some aspects, various thickeners, flavorings, diluents, emulsifiers, dispersing aids or binders and the like are added. The compositions of the present invention may contain any such additional ingredients so as to provide the composition in a form suitable for administration. The final amount of compound in the formulations varies, but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The invention encompasses methods for treating OIC and IBS by administering at least one compound as described herein. The compounds are generally administered as a pharmacological preparation or composition. Such preparations/compositions are administered in vivo by any suitable route including but not limited to: inoculation or injection (e.g. intravenous, intraperitoneal, intramuscular, subcutaneous, intra-aural, intra-ocular, intraarticular, intramammary, and the like); or by absorption through epithelial or mucocutaneous linings; or via an enema. Other suitable means include but are not limited to: inhalation (e.g. as a mist or spray) and orally (e.g. as a pill, capsule, liquid, etc.). In preferred embodiments, the mode of administration is oral. In addition, the compositions may be administered in conjunction with other treatment modalities. For example, for treating IBS, patients may also eliminate gluten or sugars from the diet, increase fiber intake, or be prescribed e.g. anti-diarrheal, anticholinergic and/or antispasmodic medications, antibiotics, or drugs such as Alosetron (Lotronex) or Lubiprostone (Amitiza). For treating OIC, the patient may increase exercise and change eating habits, e.g. to include more fiber; various laxatives, stool softeners, etc. may be used; or medications such as methylnatrexone may be prescribed.

The amount of a compound that is administered depends on several factors, e.g. the weight, gender, age, overall health, etc. of the recipient, and is best determined by a skilled medical practitioner such as a physician. However, the amount is generally in the range of from about 1-1000 mg/kg of body weight. e.g. from about 5-500 or about 10-250 or about 15-125 mg/kg of body weight, including all integers and fractional values within these ranges.

Diseases and conditions that are treated using the compounds disclosed herein include any disease or condition that is associated with MOR and KOR activity. In particular, diseases/conditions which cause or are associated gastrointestinal disorders in which visceral pain is perceived or experienced are treated. Examples of such diseases/disorders include but are not limited to: those characterized or accompanied by pain due to colonic distension and/or colonic inflammation such as: irritable bowel syndrome; colonic inflammation and/or colitis of any type e.g. that which occurs in Parkinson's disease; ulcerative colitis; Crohn's disease; microscopic colitis; lymphocytic colitis; collagenous colitis; diversion colitis (inflammation of the colon which can occur as a complication of ileostomy or colostomy); chemical colitis (due to the introduction of harsh chemicals into the colon by an enema or other procedure); chemotherapy induced colitis; colitis caused by over-the-counter and prescription medications such as nonsteroidal anti-inflammatory drugs (NSAIDs), mycophenolate, ipilimumab, and retinoic acid; ischemic colitis; infectious colitis e.g. Clostridiumwn difficile colitis, enterohemorrhagic colitis caused by Shigella dysenteriae or the Shigatoxigenic group of Escherichia coli (STEC) and other enterohemorrhagic E. coli, colitis caused by parasitic infections e.g. by Entamoeba histolytica, etc.; indeterminate and atypical colitis, both of which may have unspecified, multiple or obscure causes, etc. Symptoms of such diseases/disorders that can be lessened or eliminated by administration of the compounds described herein include but are not limited to, for example, pain, diarrhea, constipation, and the like.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indictates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Example

This Example describes the design and synthesis of an exemplary peripherally active 6/f-N-heterocyclic naltrexamine derivative as well as in vitro and in vivo investigations of its pharmacology as a selective MOR and/or KOR antagonist. Activation of MORs on the enteric neurons of the GI tract is known to play a critical role in the development of OIC. Thus, the exemplary compound described herein and related variants thereof may be used to prevent and/or treat OIC.

### Methods

### Drugs and Chemicals.

Morphine (morphine sulfate pentahydrate salt), was procured from the National Institute of Drug Abuse (NIDA), Bethesda, Md. and made into a 10 μM stock solution by dissolving in distilled water which was further diluted to the desired concentrations. NAP was synthesized according to previous reports¹⁹ as the HCl salt and dissolved in distilled water to a stock concentration of 10 μM, which was further diluted with distilled water to make the targeted concentration. 1-Benzyl-17-Cyclopropylmethyl-3,14β-dihydroxy-4,5α-epoxy-6β-[(4′-pyridyl)acetamido]m orphinan (BNAP) was synthesized by the reaction of NAP as the free base with benzyl bromide in acetone at room temperature until no more precipitate was formed.

### Animals.

Male Swiss Webster mice (Harlan Laboratories, Indianapolis, Ind.) weighing 25-30 g were housed 5 to a cage in animal care quarters and maintained at 22-2° C. on a 12 h light-dark cycle. Food and water were available ad libitum. The mice were brought to a test room (22±2° C., 12 h light-dark cycle), marked for identification and allowed 18 h to recover from transport and handling. Protocols and procedures were approved by the Institutional Animal Care and Use Committee (IACUC) at Virginia Commonwealth University Medical Center and comply with the recommendations of the IASP (International Association for the Study of Pain).

### Synthesis of BNAP.

In an attempt to increase the peripheral nervous system (PNS) selectivity of NAP it was hypothesized that quaternization of the pyridal nitrogen would yield a PNS selective derivative while not negatively impacting the antinociceptive potency. Such quaternizations are typically carried out through alkylation with the appropriate alkyl halide. One point of consideration in attempting this alkylation is the presence of the basic nitrogen at the 17 position which could compete for the alkylating agent and give rise to mixtures of alkylated products. Of the alkylating agents tested, benzyl bromide would ultimately prove to be the most selective under the reaction conditions tried. Alkylation of NAP with benzyl bromide was found to give selective alkylation of the pyridyl nitrogen in a high yield with easy purification (Scheme 1). NAP (1.0 mmol), prepared according to previously described procedures¹⁹ was stirred for 5 days with benzyl bromide (5.0 mmol) in acetone. Then the reaction mixture was filtered and the solid precipitate was resuspended in fresh acetone and allowed to stir overnight at room temperature. Filtration gave BNAP as a white solid in analytically pure form. A second batch of BNAP was obtained if the filtrate was reduced to one-third its volume and allowed to stir for another 5 days. The full characterization of BNAP was carried out by HPLC, NMR and mass spectroscopy, and confirmed the chemical composition and structure (not shown).

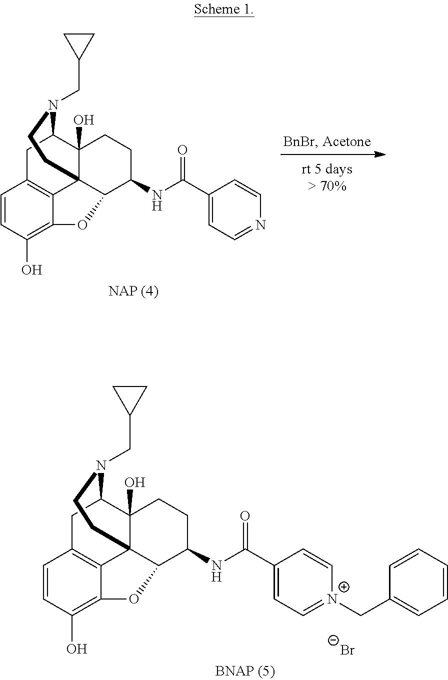

### Competitive Radioligand Binding and Functional Studies.

The details of the Radioligand Binding and Functional Studies that were used were described previously.¹⁹ Chinese hamster ovarian (CHO) cell lines were used as monoclonal opioid receptor expressing cells. The μ, δ, and κ opioid receptors were labeled with [³H]naloxone, ³H-naltrindole ([³H]NTI) and ³H-nor-Binaltorphimine ([³H]norBNI). The opioid receptor expressing cell membranes were incubated with the radioligands at different concentrations of the drug under investigation at 30° C. for 1 h. With the absence and presence of 10 μM naltrexone, specific binding was got as the difference in binding obtained. The specific binding of the drugs to the radioligand was calculated from data using linear regression analysis of Hill plots.

[³⁵S]GTPγS-binding Assays using a Low MOR-expressing CHO Cell Line: Cell membranes were incubated with varying concentrations of BNAP or 3 μM DAMGO (standard full agonist at MOR) in the presence of 10 μM GDP and 0.1 nM [³⁵S]GTPγS in assay buffer (50 mM Tris-HCl, pH 7.4, 3 mM MgCl₂, 0.2 mM EGTA and 100 mM NaCl) for 90 min at 30° C. Nonspecific binding was determined using 10 μM unlabeled GTPγS. The incubation was terminated by rapid vacuum filtration through GF/B glass fiber filters. Bound radioactivity was determined by liquid scintillation spectrophotometry. Additional methodological details of the assay were described previously.⁹

[³⁵S]-GTPγS-Binding Assays using DOR- or KOR-Expressing CHO Cell Lines: Cell membranes (10 μg protein) were incubated with 10 μM GDP and 0.1 nM [³⁵S]-GTPγS in assay buffer in the presence and absence of varying concentrations of BNAP, 3 μM SNC80 (standard full agonist at DOR) or 3 μM U50,488H (standard full agonist at KOR) for 90 min at 30° C. Nonspecific binding was determined with 10 μM unlabeled GTPγS. The reaction was terminated and bound radioactivity determined as described above and previously.⁹

### Data Analysis of [³⁵S]-GTPγS-Binding Assays.

All samples were assayed in triplicate and repeated at least twice for a total of 3 independent determinations. Results were reported as mean values±SEM. Concentration-effect curves were fit by nonlinear regression to a one-site binding model, using GraphPad Prism software, to determine EC₅₀ and Eₘₐₓ values. IC₅₀ values were obtained from Hill plots and analyzed by linear regression using Microsoft Excel software. Binding Kᵢ values were determined from IC₅₀ values using the Cheng-Prusoff equation: Kᵢ=IC₅₀/l+([L]/K_{D}), where [L] is the concentration of competitor and K_{D} is the K_{D} of the radioligand.

### Docking Studies.

The structure of BNAP was sketched using SYBYL-X 2.0. After energy minimization (10,000 iterations), the Gasteiger-Hückel charges of BNAP were assigned using TAFF. The docking study was conducted via GOLD 5.1 with standard default settings. The defined binding sites included all atoms within 10 Å of the α-carbon atom of Asp^{3 25} for the crystal structures of MOR, KOR and DOR. The best docked solution was selected based on the fitness scores and the binding orientation of each ligand within the binding cavity. In order to remove clashes and minimize strain energy, the combined receptor-ligand structures were energy-minimized using the parameters described above to optimize the interactions between ligand and receptor within the binding pocket.

### Tail Immersion Test.

The warm water tail-immersion test was performed according to previously described methods using a water bath with the temperature maintained at 56±0.1° C.²⁰ Briefly, before giving the mice injections, a baseline (control) latency was determined. Only mice with a control reaction time of 2 to 4 s were used. The average baseline latency for these experiments was 3.0±0.1 s. The test latency after drug treatment was assessed at the appropriate time, and a 10 s maximal cutoff time was imposed to prevent tissue damage. Antinociception was quantified according to established procedures as the percentage of maximum possible effect (% MPE), which was calculated as follows: % MPE=[(test latency−control latency)/(10−control latency)]×100.²¹ Percentage MPE was calculated for each mouse, using at least six mice per group.

### Intracerebroventricular Injections.

Intracerebroventricular (ICV) injections were performed as described previously.²² Mice were anesthetized with 2.5% isoflurane and a horizontal incision was made in the scalp. A needle was inserted to a depth of 3 mm into the lateral ventrical (2 mm rostral and 2 mm lateral at a 45° angle from the bregma). At intervals, 5 μL injections of drug or vehicle were made using this guide insertion to the same depth using a needle with a guard in nonanesthetized animals.^{22, 23} Animals underwent the anesthetized surgery in the morning of the experiment and were then injected with drug at intervals indicated in the text without additional anesthesia. Immediately after testing, the animals were euthanized to minimize any type of distress, according to IACUC guidelines.

### Preparation of Colon Circular Muscle for Isometric Tension Recordings.

Mice were euthanized by cervical dislocation. The colon was dissected, flushed of its contents, and trimmed of mesentery. Segments of the distal colon (approximately 1 cm from anus) and proximal colon (approximately 1.5 cm from distal portion) were removed and placed in a dissecting dish containing Krebs solution (118 mM NaCl, 4.6 mM KCl, 1.3 mM NaH₂PO₄, 1.2 mM MgSO₄, 25 mM NaHCO₃, 11 mM glucose, and 2.5 mM CaCl₂) bubbled with 95% O₂ and 5% CO₂. The tissues, 0.5 cm in length, were suspended vertically along the axis of the circular muscle with a metal triangle tied to a hook under 1 g of passive tension in 15 mL siliconized organ baths. The tissues were allowed to equilibrate for 60-90 min prior to drug exposure, with the Krebs solution changed every 10 min for the first 40 min. Prior to administering the selected antagonist a control run was conducted to ensure the preparations were responding to morphine. Tissues not responding to morphine in the control run were discarded. Following the control run, tissue samples were washed with Krebs every 10-15 min for 1.5-2.0 hours until values returned to baseline levels. Following this, the antagonist was added, allowed to incubate with the tissue for 15 min and a second dose response curve to morphine was conducted. Isometric contractions were recorded by a force transducer (GR-FT03; Radnoti, Monrovia, Calif.) connected to a personal computer using Acknowledge 382 software (BIO-PAC Systems, Inc., Santa Barbara, Calif.).

### Charcoal Meal Test for Gastrointestinal Transit Analysis.

Forty-eight hours before testing, mice were placed in cages with raised mesh wire to suspend them above their bedding and prevent ingestion of feces or bedding. The animals were habituated for 24 h in the presence of food and water and then fasted for 24 h with free access to water as previously reported.²⁴ This time frame was chosen to deplete the intestine and colon of any feces. To maintain caloric intake and to avoid hypoglycemia, mice had access to a sugar water solution consisting of a final concentration of 5% dextrose for the first 8 h of the fasting period. Mice were treated with either saline (10 μL/g s.c.) or morphine (10 mg/kg s.c.), and 20 min later they were given an oral gavage consisting of 5% aqueous suspension of charcoal in a 10% gum Arabic solution. At 30 min after the administration of the charcoal meal, the mice were euthanized by cervical dislocation, and the small intestine from the jejunum to the cecum was dissected and placed in cold saline to stop peristalsis. The distance traveled by the leading edge of the charcoal meal was measured relative to the total length of the small intestine, and the percentage of intestinal transit for each animal was calculated as percentage transit (charcoal distance)/(small intestinal length)×100. This is referred to as intestinal transit in the text.

### Colonic Bead Expulsion Assay.

Mice were habituated and fasted as described above for the gastrointestinal transit analysis. Mice were given an injection of either saline (10 μL/g b·wt.) or morphine (10 mg/kg s.c.). At 20 min post-injection, animals were anesthetized with isoflurane (1-2 min) to insert a single 2-mm glass bead into the distal colon at a distance of 3 cm from the anus. Bead insertion was accomplished using a glass rod with a fire-polished end to avoid tissue damage and marked at 3 cm.²⁵ After bead insertion, mice were placed in individual cages and the time to bead expulsion was monitored. Animals were monitored for a maximum of 2 h unless bead expulsion occurred sooner.

### Guinea Pig Longitudinal Muscle Myenteric Plexus Strips for Isometric Tension Recordings.

Male albino guinea pigs (350-450 g) were euthanized by asphyxiation with CO₂. A segment of ileum was removed and placed in a dissection bath filled with pre-oxygenated Krebs' solution (118 mM NaCl, 4.6 mM KCl, 1.3 mM NaH₂PO₄, 1.2 mM MgSO₄, 25 mM NaHCO₃, 11 mM glucose, and 2.5 mM CaCl₂). Longitudinal Muscle Myenteric Plexus (LMMP) strips were prepared as previously described.²⁶ Electrical field stimulation (EFS) (50 V, 7.5 Hz, unless stated otherwise) was applied through concentric electrodes over longitudinal muscle or L-type stimulating electrodes over circular muscle strips to produce neurogenic contractions/relaxations. Drugs were added in single or cumulative doses depending on the assay to determine their inhibitory effects on the neurogenic responses.

### Measurement of BNAP Antagonism of Acetic Acid Induced Writhing.

To determine whether BNAP provides KOR mediated analgesia acetic acid-induced abdominal stretches were counted in mice. Swiss Webster mice were allowed to acclimate in individual testing cages for at least 10 min. Following the acclimation period the vehicle or BNAP was administered via s.c injection at the specified dose. After waiting for 10 min the mice were injected intraperitoneally with 10 mL/kg 0.6% (wt/vol) acetic acid and returned to their observation chambers. After the first 3 min, the number of stretches and abdominal contractions were counted for 15 min. All values shown for abdominal stretching experiments represent separate groups of mice (n=4-8) as each animal was tested only once and euthanized immediately after testing.

In order to confirm the involvement of the KOR in the analgesia produced by BNAP in the Acetic Acid Induced Writhing test, this test was performed in the presence of the KOR-specific antagonist nor-Binaltorphimine 2HCl (nor-BNI). According to previous literature, nor-BNI antagonist activity reaches optimal selectivity and efficacy in the writhing test at 24 hours post administration.^{27, 28} These animals were injected sub-cutaneously with either 10 mg/kg nor-BNI or saline 24 hours prior to initiation of the writhing test. The writhing test was then performed as described above (n=5-6).

### Data Analysis.

Contractile responses after repeated administration of morphine were analyzed by taking the integrated responses between doses. Data are presented as the mean±S.E.M, values of P≤0.05 were considered significant. Effective concentration of agonists to produce 50%-maximal response [reported as negative log (EC₅₀) or pD₂] was calculated by nonlinear regression, and data were analyzed by appropriate statistical tools using GraphPad Prism software (Graph-Pad Software Inc.).

Results and Discussion It is generally agreed that activation of MORs on the enteric neurons of the GI tract plays a critical role in the development of OIC. In this study, An exemplary peripherally selective 6β-N-heterocyclic naltrexamine derivative was designed and synthesized and its pharmacology was investigated in vitro and in vivo as a selective MOR antagonist. BNAP was synthesized in one step via alkylation with benzyl bromide in high yield and purity. Having synthesized and structurally characterized BNAP, radioligand binding and receptor functional studies were carried out at MOR, KOR and DOR to determine the impact of the benzyl group on binding affinity, selectivity, and functional activity at each of the three opioid receptors. The binding affinity and functional activity for BNAP and NAP at each receptor was determined as previously described⁹ and the results are shown in Table 1.

TABLE 1
Binding Affinity and ³⁵S-GTP[γS] assay results for BNAP and NAP^{9b} at MOR,
KOR and DOR. The values are the mean ± SEM of three independent experiments.
[³H]Naloxone, [³H]NTI, and [³H]nor-BNI were used to label MOR, DOR, and KOR,
respectively, unless otherwise stated. The percentage stimulation to agonist is the Eₘₐₓ
of the compound compared to that of a full agonist (normalized to 100%): DAMGO
for MOR, SNC80 for DOR and U50,488H for KOR.

| Compound | Receptor | Kᵢ (nM) ± SEM | Selectivity | EC₅₀ (nM) | % max of agonist |
|---|---|---|---|---|---|
| BNAP | MOR | 0.76 ± 0.09 | — | 4.8 ± 0.6 | 14.6 ± 0.9 |
| | KOR | 3.46 ± 0.05 | 4.6 (κ/μ) | 2.9 ± 1.1 | 45.9 ± 1.7 |
| | DOR | 722 ± 65 | 950 (δ/μ) | 107.6 ± 69.0 | 9.2 ± 2.2 |
| NAP | MOR | 0.37 ± 0.07 | — | 1.1 ± 0.4 | 22.7 ± 0.8 |
| | KOR | 60.72 ± 5.58 | 163 (κ/μ) | 28.8 ± 14.4 | 45.5 ± 4.4 |
| | DOR | 277.51 ± 7.97 | 747 (δ/μ) | 15.2 ± 15.2 | 10.2 ± 3.1 |

As can be seen, BNAP maintained sub-nanomolar affinity for MOR with a Ki=0.76±0.09 nM compared with that of the parent compound NAP (Ki=0.37±0.07). BNAP showed a >900 fold selectivity for the MOR over the DOR. Interestingly its selectivity over the KOR was less than the parent compound NAP. Ligand-stimulated [³ S]GTPγS binding was then utilized to determine the relative efficacy of BNAP to activate MOR, DOR and KOR. At the MOR, BNAP showed low agonist efficacy with a response only 14.6±0.9% of the maximum response for the full agonist DAMGO (NAP, 22.72±0.84%), with an EC₅₀ value of 4.84±0.60 nM (NAP, 1.14±0.38 nM). Similarly at the DOR, BNAP behaved as a low efficacy agonist with a % maximum response at 9.2±2.2% (NAP, 10.2±3.1%) when compared to the full agonist SNC80, and also low potency to activate the DOR (EC₅₀=107.6±69.0 nM; NAP, 15.2±15.2 nM). In conjunction with the lower selectivity of BNAP at KOR, BNAP also displayed partial agonist activity at KOR with a % maximum response at 45.9±1.7% when compared to the full agonist U50,488H (NAP=45.5±4.4%). The potency for BNAP relative to NAP was greater at KOR, with an EC₅₀ value of 2.9±1.1 nM compared to 28.8±14.4 nM for NAP. Interestingly, despite the 4.6-fold lower binding affinity of BNAP for the KOR relative to the MOR, this compound had approximately equal potency to activate these two receptors in the functional assay. Compared to NAP, the selectivity profile for BNAP was changed from MOR selective to MOR/KOR dually selective.

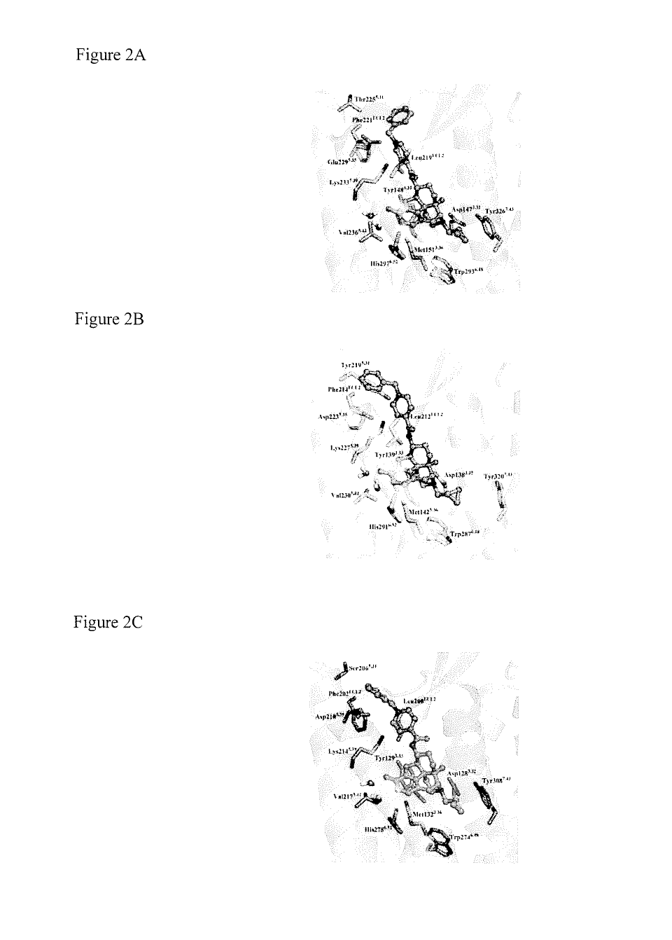

To help understand possible reasons for the observed changes in selectivity of BNAP over the parent compound, docking studies of BNAP in the MOR, DOR and KOR antagonist-bound crystal structures were undertaken. As seen in FIG. 2A-C, the binding pocket for the morphinan skeleton of BNAP across the three opioid receptors was highly conserved. However, differences in the residues surrounding the alkylated pyridine provided insight into potential changes in affinity and selectivity. Within the MOR, Glu229 may interact with the positively charged nitrogen atom of the pyridyl ring via ionic interactions. Within the KOR and DOR Asp223 and Asp210 occupy these positions respectively. The shorter length of the aspartate residue would not allow for such stabilizing ionic interactions. However, in KOR plausible hydrogen bonding interactions between the pyridyl nitrogen and Tyr219 could provide similar stabilization as that seen in MOR. These studies showed that the binding pocket of MOR, DOR, and KOR can accommodate the added benzyl ring; however, the introduction of the positive charge on the pyridyl nitrogen may be responsible for the reduced MOR/KOR selectivity due to the now possible hydrogen bonding interactions within the KOR.

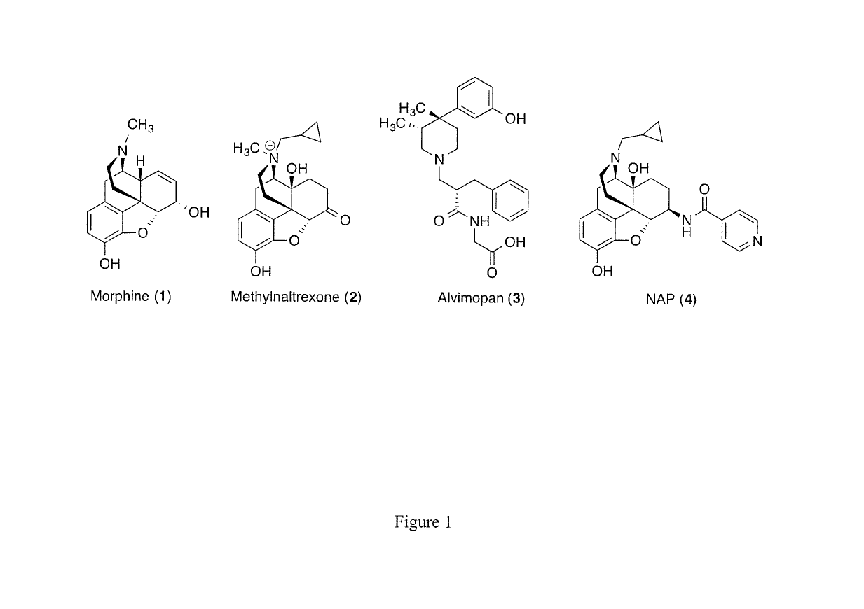

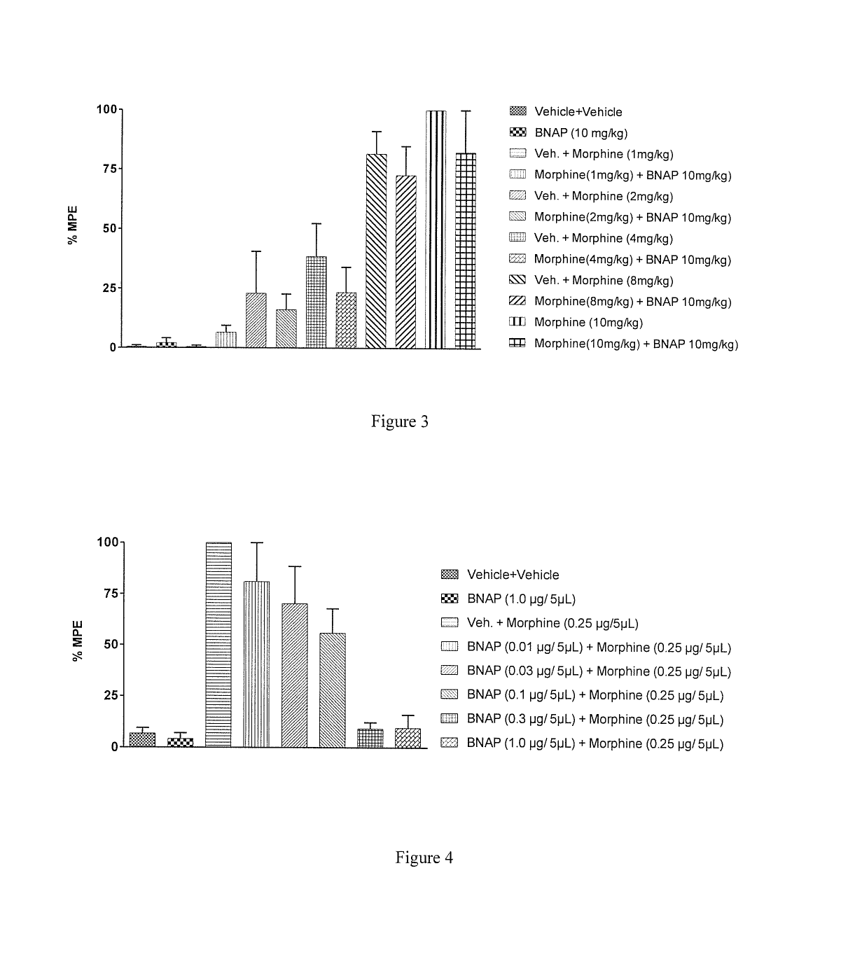

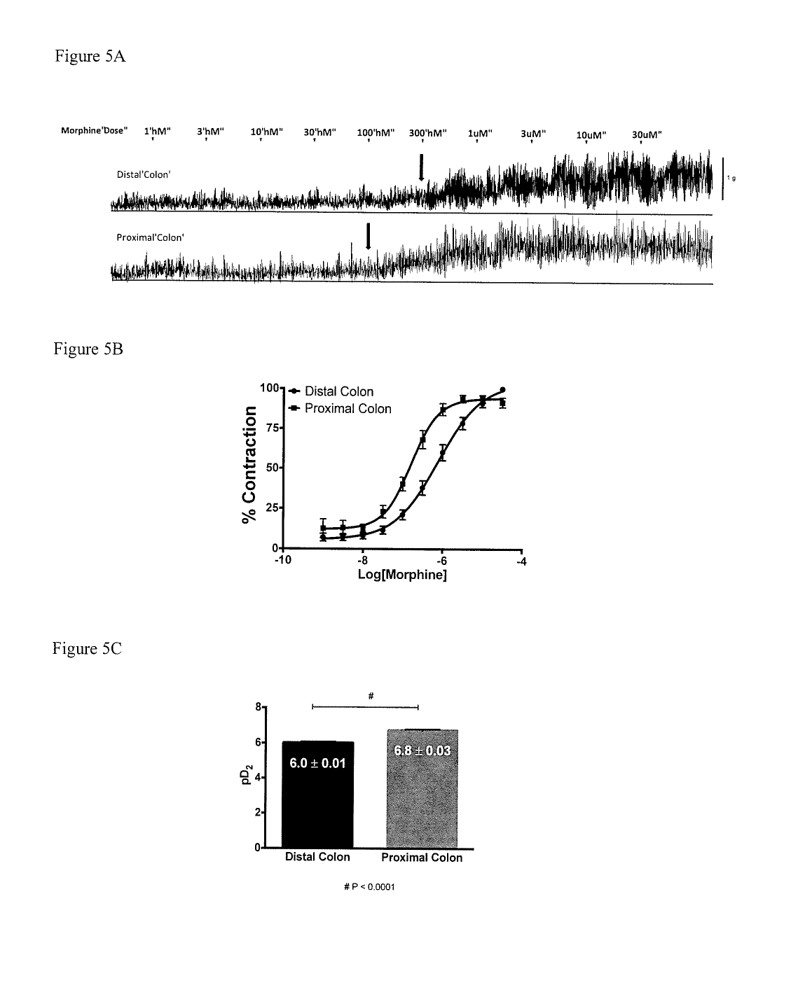

To further examine the pharmacology of BNAP, a combined in vivo and in vitro approach was taken. We first determined the effects of BNAP on morphine (1, FIG. 1) antinociception following two routes of administration. This set of experiments was designed to give us insight into the potency of BNAP within the CNS by measuring its effect on morphine antinociception in the warm water tail flick assay. As shown in FIG. 3 at 10 mg/kg BNAP s.c. alone did not produce significant antinociceptive effects whereas morphine dose dependently induced antinociception. BNAP at 10 mg/kg did not significantly antagonize morphine antinociception in the warm water tail flick assay at any of the morphine doses tested. These results supported our hypothesis that quaternization of the pyridyl nitrogen limited activity of BNAP to the periphery. To further determine if BNAP behaves as an MOR antagonist, BNAP was administered via intracerebroventricular (ICV) injection (thus directly into the CNS) before assessing activity in the warm-water tail-flick assay. Under this experimental paradigm BNAP alone at 1.0 μg/5 μL did not produce significant antinociception when compared to morphine alone (FIG. 4). A dose dependent reversal of morphine antinociception was observed with increasing concentrations of BNAP with complete reversal occurring at 0.3 μg/5 μL ICV of BNAP. These results are consistent with the in vitro functional assay data identifying BNAP as an MOR antagonist. These data in conjunction with the subcutaneously administered BNAP experiments supported our hypothesis that quaternization of the pyridyl nitrogen with the benzyl group limits CNS penetration of BNAP. BNAP did not antagonize morphine-induced thermal antinociception when administered subcutaneously, but reversed morphine antinociception when administered via intracerebroventricular (ICV) route. Beyond antagonism of morphine-induced thermal antinociception, another important observation from these studies was lack of thermal antinociception displayed by BNAP alone irrespective of the route of administration. This result was significant as the radioligand binding and in vitro functional studies showed BNAP to have moderate potency and efficacy as a KOR agonist considering the fact that KOR agonists are typically known to have psychotomimetic side effects that accompany their analgesic properties.¹⁰

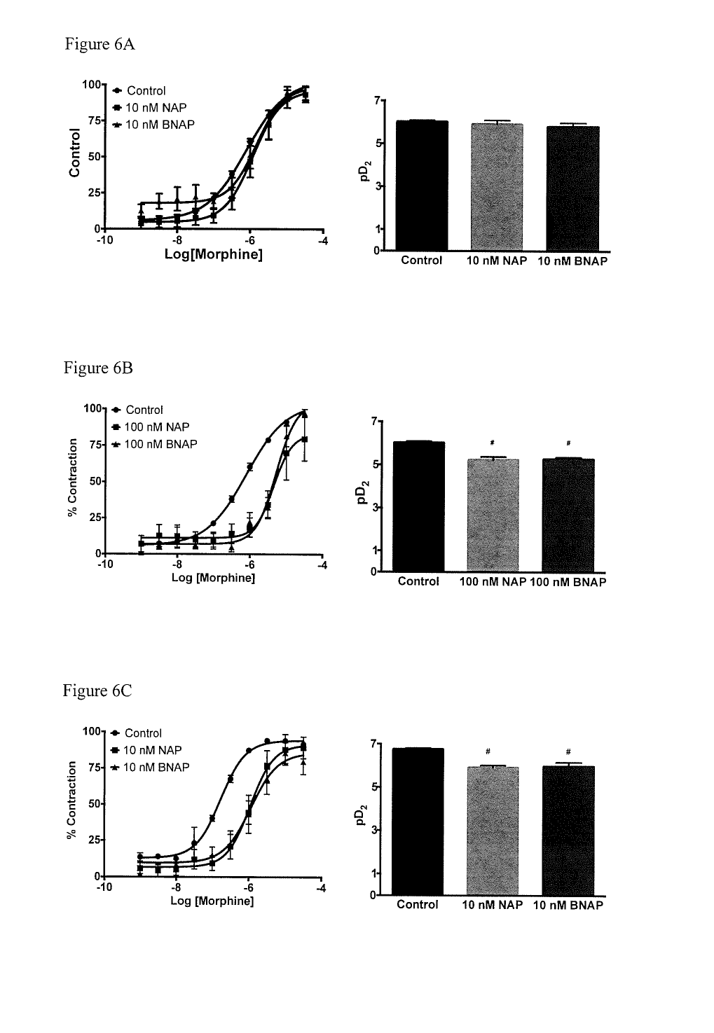

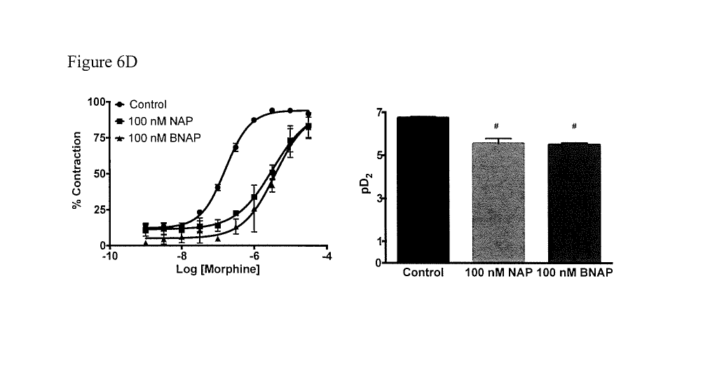

Recently, there has been a renewed interest in understanding the regional differences in responsiveness of the rodent GI tract to various drug classes.^{11a, 12} Ono et al. have recently suggested that regions more sensitive to the effects of morphine play a key role in the development of OIC.^{11a} Radioligand binding and in vitro functional studies showed that NAP and BNAP had comparable affinity and similar efficacy for the MOR in the [³'S]GTPγS-binding assay. Thus, as expected, BNAP should demonstrate similar inhibitory potency as that of the parent compound (NAP) in the isolated tissue preparations. One of the approaches used to study the effects of opioid ligands on the GI tract is the examination of isometric tension recordings from isolated tissues preparations, which our laboratory has utilized extensively to study the development of opioid tolerance in the mouse GI tract.^{11b, 13} We have previously shown that morphine induced contractions of the circular muscle and work by Ono has suggested that the circular muscle contractions played a key role in the constipation effects of opioids.¹¹ Therefore we examined the effects of BNAP on morphine induced circular muscle contractions of the mouse colon. As expected, morphine dose dependently induced contractions in both the distal and proximal colon sections (FIG. 5A). The proximal colon responded to morphine at concentrations ten times lower than those that produced equal effects in the distal colon, suggesting that this portion of the colon is more sensitive to morphine's effects (FIG. 5B). The pD₂ value for morphine in naive tissue in the distal colon was 6.0±0.1 which were consistent with previous findings.^{11b} In the proximal colon the morphine pD₂ was 6.8±0.1, nearly ten-fold difference in morphine sensitivity. Having observed these differences in response to morphine in the distal and proximal sections, the functional activity of BNAP was examined in both preparations and compared to the parent compound NAP to determine possible differences in sensitivity to these antagonists. Neither BNAP nor NAP alone at concentrations up to 30 μM induced contractions in the tissue preparations. Accordingly, each antagonist (BNAP or NAP) was evaluated at three concentrations: 1, 10 and 100 nM in both distal and proximal colon preparations. At 1 nM, neither NAP nor BNAP showed significant inhibition of morphine-induced contractions when compared to controls in the distal or proximal colon preparations (data not shown). In the distal colon at 10 nM neither NAP nor BNAP showed significant antagonism of morphine effects (FIG. 6A) while at 100 nM significant antagonism of morphine affects were observed for both as noted by the reduction in morphine pD₂ (NAP 5.3 and BNAP 5.2 FIG. 6B). In the proximal colon at 10 nM NAP and BNAP showed equal antagonism of morphine reducing the pD₂ for morphine to 5.9 (FIG. 6C). Increasing the concentration of NAP and BNAP to 100 nM showed no further reduction of the morphine pD₂ suggesting antagonism was near maximal at 10 nM. In all, at all of the concentrations tested, BNAP and NAP showed comparable activities for antagonizing morphine induced contractions of the mouse distal and proximal colon. We observed that the proximal colon is approximately 10-fold more sensitive to morphine than the distal colon. Regional difference in morphine's effect along the GI tract have been previously demonstrated.^{1b}

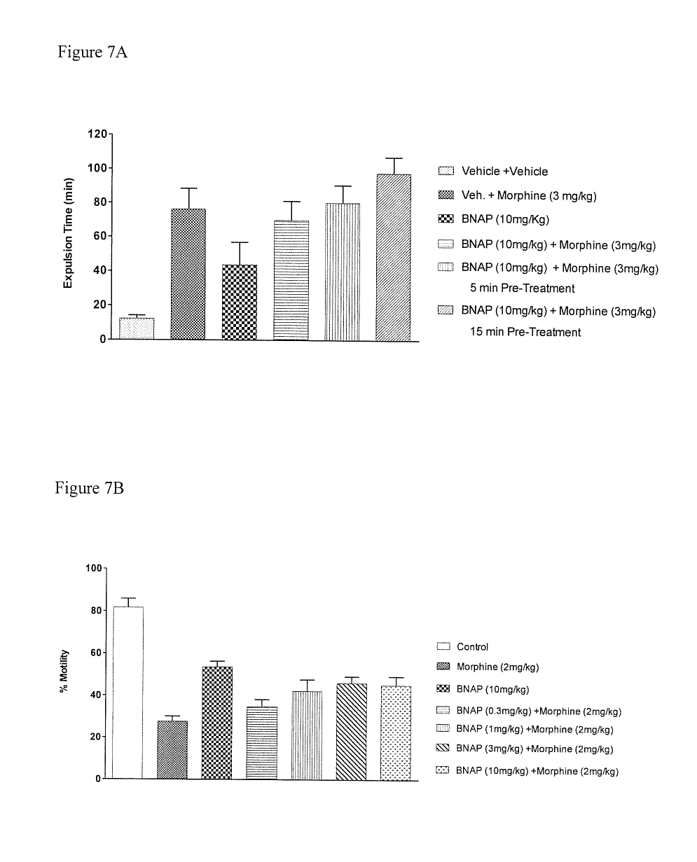

Having confirmed antagonist activity of BNAP in the circular muscle preparations as well as limited CNS activity, we next examined the effect of BNAP on GI motility in mice treated acutely with morphine using the charcoal gavage and colonic bead expulsion assays (FIG. 7A-B). We first examined the effects of BNAP on colonic motility. In naive mice the average bead expulsion time was 17 min. Following exposure to morphine at 3 mg/kg, bead expulsion time was increased to 75 min. It was anticipated that administration of BNAP would decrease expulsion time. Surprisingly, when administered alone BNAP at 10 mg/kg increased bead expulsion time to 43 min. When administered in the presence of morphine either concurrently or as a pretreatment 5 or 15 min before morphine, no significant antagonism of morphine's effects was observed. Similar observations were observed that BNAP reduced intestinal motility 1.5-fold compared to the 3-fold decrease by morphine when examined in the charcoal gavage intestinal motility assay. Thus, unlike the parent compound NAP⁷, BNAP was unable to reverse morphine induced inhibition of intestinal motility. These in vivo data were unexpected and seemed contradictory to those observed in the in vitro systems. One possible reason for this difference could be the partial agonist activity of BNAP at the KOR.

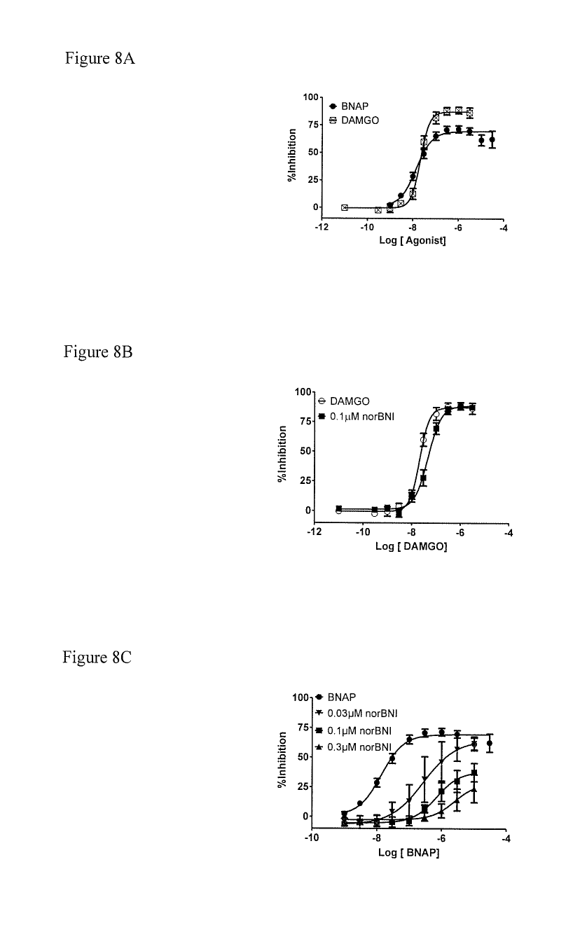

The in vivo data described above suggested that: 1) BNAP behaves differently in longitudinal muscle than in circular muscle and 2) the partial agonism of BNAP at KOR may prevent MOR antagonism in vivo. Fichna recently reported that KOR agonist Salvinorin A reduced colonic motility in rodent models.¹⁴ In light of this finding we suspected that the seemingly dichotomous nature of BNAP was the result of its partial agonsim at KOR acting on longitudinal muscle. To test this hypothesis we investigated the effects of BNAP on Electrical Field Stimulated (EFS) contractions in Longitudinal Muscle Myenteric Plexus (LMMP) preparations from the guinea pig ileum. Studies by others and us have shown that MOR agonists inhibited EFS contractions in a dose dependent manner; however, little is known about the effects of KOR agonists on EFS contractions of LMMP preparations. As shown in FIG. 8A, BNAP dose dependently inhibited EFS contractions in LMMP preparations with comparable potency to the selective MOR agonist DAMGO (pIC₅₀=7.67±0.03, pIC₅₀ BNAP=7.86±0.07). That result is in agreement with its KOR partial agonist behavior seen in [³⁵S]GTPγS-binding Assays. To establish the potential role of KOR in this response, the tissue was pretreated with the selective KOR antagonist nor-BNI 15 min prior to agonist exposure. FIG. 8B showed that nor-BNI at 0.1 μM did not significantly effect DAMGO inhibition of EFS contractions. On the other hand, nor-BNI dose dependently shifted the BNAP concentration-response curve to the right, suggesting the inhibitory effects of BNAP on GI motility as observed in the in vivo studies were in part mediated via activation of KOR (FIG. 8C).

As stated previously, there are conflicting reports concerning the effect of KOR agonist on GI motility.^{10, 14} In humans, Asimadoline, a peripheral kappa agonist, did not alter GI transit.¹⁵ We found that the inhibitory effects of BNAP on longitudinal muscle were blocked by the selective KOR antagonist nor-BNI suggesting that impairment of GI motility by BNAP result at least in part from KOR agonism. Additionally, this study sheds light on the role that the KOR plays in mouse GI motility and our results are consistent with the work of Fichna, which has shown that the full KOR agonist Salvinorin A can reduce GI motility in mouse models.¹⁴ These results were also surprising as BNAP showed no activity in thermal antinociception studies when administered via ICV injection. This may suggest significant differences in sensitivity of the KORs expressed in the periphery vs centrally.

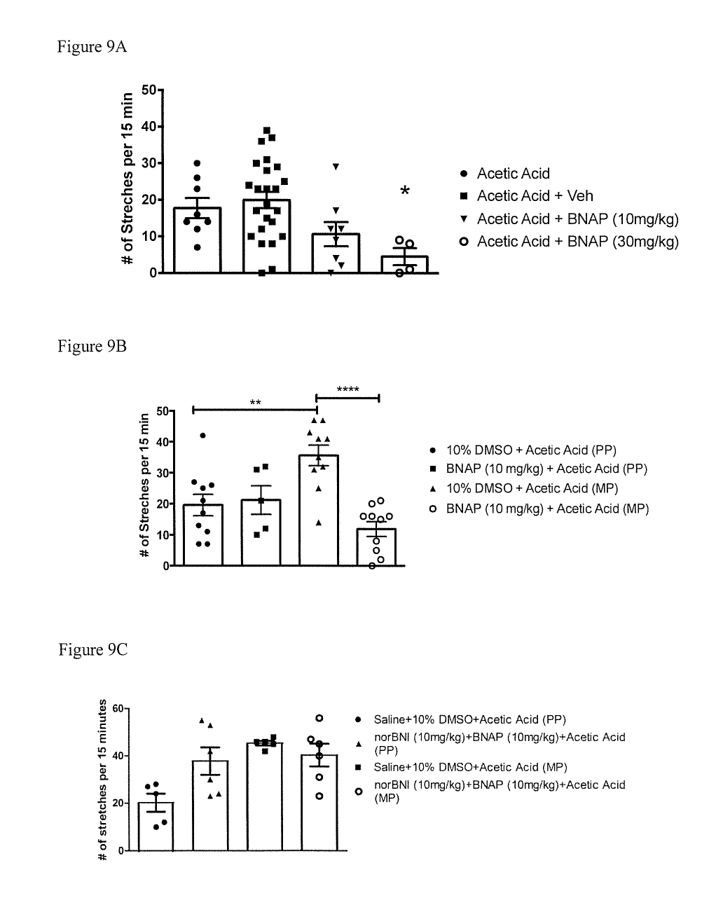

Within the past few years a renewed interest has been shown in the development of peripherally selective KOR agonists.¹⁶ Such agonists are of interest because they display analgesic activity while avoiding the negative side effects associated with MOR agonists.¹⁰ To further investigate KOR mediated effects of BNAP, a visceral pain model was utilized. Given the mixed MOR/KOR pharmacological profile observed by BNAP, we proposed that this exemplary ligand may serve as a novel analgesic for the treatment of visceral pain without side effects associated with centrally active KOR agonists. Therefore, the acetic acid writhing assay was used to determine the effect of systemically administered BNAP on nociception by s. c. In this assay, BNAP dose dependently reduced the number of writhes (FIG. 9A). We also examined the effects of BNAP in a hypernociceptive model of visceral pain. We have previously shown that mice that were rendered tolerant to the analgesic effects of morphine by 5-day pellet implantations exhibit a two-fold increase in the writhing responses. When tested in this assay BNAP was active at three fold lower concentration (FIG. 9B) compared to morphine. Recent evidence suggests that KOR agonists can decrease the overall perception of pain due to colonic distension in irritable bowel syndrome patients. The effects of Asimadomine, a peripherally active kappa agonist in clinical trials, are more pronounced in models of colonic inflammation and may reflect changes/increases in KOR in disease states. Our findings that BNAP significantly reduced abdominal stretches in chronic morphine mice compared to naive mice supports this hypothesis. In order to further confirm this, we performed the acetic acid writhing test in the presence of the selective KOR antagonist, nor-BNI (FIG. 9C). Antagonism of the peripheral KOR effectively blocked the analgesia produced by BNAP in the morphine pelleted animals. We have previously shown that hypernociception occurs in chronic morphine treated mice¹⁷ and may be due to bacterial translocation following chronic morphine induced breakdown of the epithelial barrier.¹⁸ The kappa-agonist activity results in a compound that reduces visceral pain sensation and in combination with peripheral mu opioid receptor antagonism has potential use in functional GI disorders such as irritable bowel syndrome.

In summary, we have shown that the introduction of a permanent charge in NAP derivatives (as in the exemplary compound BNAP) limits their activity to the periphery while maintaining affinity for both the MOR and KOR. Through in vitro and in vivo studies, it was determined that BNAP has mixed pharmacology acting as a MOR antagonist and KOR partial agonist. BNAP did not reverse morphine's effects on GI motility, however it was effective in reducing pain responses in the writhing assay. In particular in hypernocicepetion models of visceral pain BNAP was active at concentrations three-fold lower than in morphine naive animals. Together these data show BNAP to be useful in the treatment of irritable bowel syndrome in patients suffering from visceral pain that is accompanied by both constipation and diarrhea.

### REFERENCES

(1) (a) Brock, C., Olesen, S. S., Olesen, A. E., Frokjaer, J. B., Andresen, T., Drewes, A. M. (2012) Opioid-induced bowel dysfunction: pathophysiology and management. Drugs 72, 1847-1865. (b) Akbarali, H. I., Inkisar, A., Dewey, W. L. (2014) Site and mechanism of morphine tolerance in the gastrointestinal tract. Neurogastroenterol Motil. 26, 1361-1367. (c) Grond, S., Zech, D., Diefenbach, C., Bischoff, A., (1994) Prevalence and pattern of symptoms in patients with cancer pain: a prospective evaluation of 1635 cancer patients referred to a pain clinic. Journal of pain and symptom management 9, 372-382. (d) Zech, D. F., Grond, S., Lynch, J., Hertel, D., Lehmann, K. A. (1995) Validation of World Health Organization Guidelines for cancer pain relief: a 10-year prospective study. Pain 63, 65-76. (e) Thompson, A. R., Ray, J. B. (2003) The importance of opioid tolerance: a therapeutic paradox. Journal of the American of Surgeons 196, 321-324.
(2) Camilleri, M. (2011) Opioid-induced constipation: challenges and therapeutic opportunities. Am. J. Gastroenterol. 106, 835-842, quiz 843.
(3) Wood, J. D., Galligan, J. J. (2004) Function of opioids in the enteric nervous system. Neurogastroenterol. Motil. 16, 17-28.
(4) Camilleri, M., Rothman, M., Ho, K. F., Etropolski, M. (2011) Validation of a bowel function diary for assessing opioid-induced constipation. Am. J. Gastroenterol. 106, 497-506.
(5) Becker, G., Galandi, D., Blum, H. E. (2007) Peripherally acting opioid antagonists in the treatment of opiate-related constipation: a systematic review. J. Pain Symptom Manage. 34, 547-765.
(6) (a) Schmidt, W. K. (2001) Alvimopan* (ADL 8-2698) is a novel peripheral opioid antagonist. Am. J. Surg. 182, 27S-38S; (b) Lee, J. M., Mooney, J. (2012) Methylnaltrexone in treatment of opioid-induced constipation in a pediatric patient. Clin. J. Pain 28, 338-341.
(7) Yuan, Y., Stevens, D. L., Braithwaite, A., Scoggins, K. L., Bilsky, E. J., Akbarali, H. I., Dewey, W. L., Zhang, Y. (2012) 6beta-N-Heterocyclic substituted naltrexamine derivative NAP as a potential lead to develop peripheral mu opioid receptor selective antagonists. Bioorg. Med. Chem. Lett. 22, 4731-4734.
(8) Zhang, Y., Williams, D. A., Zaidi, S. A., Yuan, Y., Braithwaite, A., Bilsky, E. J., Dewey, W. L., Akbarali, H. I., Streicher, J. M., Selley, D. E. (2016) 17-Cyclopropylmethyl-3, 14beta-dihydroxy-4,5alpha-epoxy-6beta-(4′-pyridylcarboxami do)morphinan (NAP) Modulating the Mu Opioid Receptor in a Biased Fashion. ACS chemical neuroscience, DO1: 10.1021/acschemneuro.5b00245.
(9) (a) Thompson, C. M., Wojno, H., Greiner, E., May, E. L., Rice, K. C., Selley, D. E. (2004) Activation of G-proteins by morphine and codeine congeners: insights to the relevance of 0- and N-demethylated metabolites at mu- and delta-opioid receptors. J. Pharmacol. Exp. Ther. 308, 547-554. (b) Yuan, Y. Y., Li, G., He, H. J., Steveds, D. L., Kozak, P., Scoggins, K. L., Mitra, P., Gerk, P. M., Selley, D. E., Dewey, W. L., Zhang, Y. (2011) Characterization of 6α- and β-N-heterocyclic substituted naltrexamine derivatives as novel leads to development of mu opoiod recepter selevtive antagonists. ACS. Chem. Neurosci. 2, 346-351.
(10) Riviere, P. J. (2004) Peripheral kappa-opioid agonists for visceral pain. Br. J. Pharmacol 141, 1331-1334.
(11) (a) Ono, H., Nakamura, A., Matsumoto, K., Horie, S., Sakaguchi, G., Kanemasa, T. (2014) Circular muscle contraction in the mice rectum plays a key role in morphine-induced constipation. Neurogastroenterol. Motil. 26, 1396-1407. (b) Ross, G. R., Gabra, B. H., Dewey, W. L., Akbarali, H. I. (2008) Morphine tolerance in the mouse ileum and colon. J. Pharmacol. Exp. Ther. 327, 561-572.
(12) Gursoy, N., Durmus, N., Bagcivan, I., Sarac, B., Parlak, A., Yildirim, S., Kaya, T. (2008) Investigation of acute effects of aflatoxin on rat proximal and distal colon spontaneous contractions. Food. Chem. Toxicol. 46, 2876-2880.
(13) Maguma, H. T., Dewey, W. L., Akbarali, H. 1. (2012) Differences in the characteristics of tolerance to mu-opioid receptor agonists in the colon from wild type and beta-arrestin2 knockout mice. Eur. J. Pharmacol. 685, 133-140.
(14) Fichna, J., Schicho, R., Andrews, C. N., Bashashati, M., Klompus, M., McKay, D. M., Sharkey, K. A., Zjawiony, J. K., Janecka, A., Storr, M. A. (2009) Salvinorin A inhibits colonic transit and neurogenic ion transport in mice by activating kappa-opioid and cannabinoid receptors. Neurogastroenterol. Motil. 21, 1326-e128.
(15) Delgado-Aros, S., Chial, H. J., Camilleri, M., Szarka, L. A., Weber, F. T., Jacob, J., Ferber, I., McKinzie, S., Burton, D. D., Zinsmeister, A. R. (2003) Effects of a kappa-opioid agonist, asimadoline, on satiation and GI motor and sensory functions in humans. Am. J. Physiol. Gastrointest. Liver Physiol. 284, G558-G566.
(16) Bourgeois, C., Werfel, E., Galla, F., Lehmkuhl, K., Torres-Gomez, H., Schepmann, D., Kogel, B., Christoph, T., Strassburger, W., Englberger, W., Soeberdt, M., Huwel, S., Galla, H. J., Wunsch, B. (2014) Synthesis and pharmacological evaluation of 5-pyrrolidinylquinoxalines as a novel class of peripherally restricted kappa-opioid receptor agonists. J. Med. Chem. 57, 6845-6860.
(17) Ross, G. R., Gade, A. R., Dewey, W. L., Akbarali, H. I. (2012) Opioid-induced hypernociception is associated with hyperexcitability and altered tetrodotoxin-resistant Na⁺ channel function of dorsal root ganglia. Am. J. Physiol. Cell Physiol. 302, C1152-C1161.
(18) Meng, J., Yu, H., Ma, J., Wang, J., Banerjee, S., Charboneau, R., Barke, R. A., Roy, S. (2013) Morphine induces bacterial translocation in mice by compromising intestinal barrier function in a TLR-dependent manner. PLoS One 8, e54040.
(19) Li, G., Aschenbach, L. C., He, H., Selley, D. E., Zhang, Y. (2009) 14-O-Heterocyclic-substituted naltrexone derivatives as non-peptide mu opioid receptor selective antagonists: design, synthesis, and biological studies. Bioorg. Med. Chem. Lett. 19, 1825-1829.
(20) Coderre, T. J., Rollman, G. B. (1983) Naloxone hyperalgesia and stress-induced analgesia in rats. Life Sci. 32, 2139-2146.
(21) Harris, L. S., Pierson, A. K. (1964) Some Narcotic Antagonists in the Benzomorphan Series. J. Pharmacol. Exp. Ther. 143, 141-148.
(22) Pedigo, N. W., Dewey, W. L., Harris, L. S. (1975) Determination and characterization of the antinociceptive activity of intraventricularly administered acetylcholine in mice. J. Pharmacol. Exp. Ther. 193, 845-852.
(23) (a) Hull, L. C., Llorente, J., Gabra, B. H., Smith, F. L., Kelly, E., Bailey, C., Henderson, G., Dewey, W. L. (2010) The effect of protein kinase C and G protein-coupled receptor kinase inhibition on tolerance induced by mu-opioid agonists of different efficacy. J. Pharmacol. Exp. Ther. 332, 1127-1135; (b) Gabra, B. H., Bailey, C. P., Kelly, E., Smith, F. L., Henderson, G., Dewey, W. L. (2008) Pre-treatment with a PKC or PKA inhibitor prevents the development of morphine tolerance but not physical dependence in mice. Brain Res. 1217, 70-77.
(24) (a) Roy, S., Liu, H. C., Loh, H. H. (1998) mu-Opioid receptor-knockout mice: the role of mu-opioid receptor in gastrointestinal transit. Brain Res. Mol. Brain Res. 56, 281-283. (b) Raehal, K. M., Walker, J. K., Bohn, L. M. (2005) Morphine side effects in beta-arrestin 2 knockout mice. J. Pharmacol. Exp. Ther. 314, 1195-1201.
(25) (a) Raffa, R. B., Mathiasen, J. R., Jacoby, H. I. (1987) Colonic bead expulsion time in normal and mu-opioid receptor deficient (CXBK) mice following central (ICV) administration of mu- and delta-opioid agonists. Life Sci. 41, 2229-2234. (b) Martinez, V., Wang, L., Rivier, J., Grigoriadis, D., Tache, Y. (2004) Central CRF, urocortins and stress increase colonic transit via CRF1 receptors while activation of CRF2 receptors delays gastric transit in mice. J. Physiol. 556, 221-234.
(26) Kang, M., Maguma, H. T., Smith, T. H., Ross, G. R., Dewey, W. L., Akbarali, H. I. (2012) The role of beta-arrestin2 in the mechanism of morphine tolerance in the mouse and guinea pig gastrointestinal tract. J. Pharmacol. Exp. Ther. 340, 567-576.
(27) Broadbear, J. H., Negus, S. S., Butelman, E. R., de Costa, B. R., Woods, J. H. (1994) Differential effects of systemically administered nor-binaltorphimine (nor-BNI) on kappa-opioid agonists in the mouse writhing assay. Psychopharm. 115, 311-319.
(28) Negus, S. S., Morrissey, E. M., Rosenberg, M., Cheng, K., Rice, K. C. (2010) Effects of Kappa Opioids in an assay of pain-depressed intracranial self-stimulation in rats. Psychopharm. 210, 149-159.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A compound of Formula I 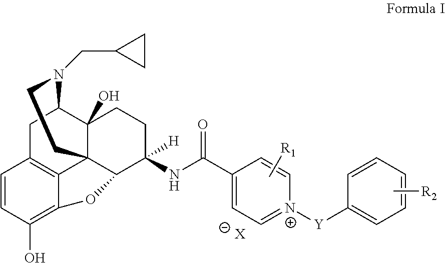
where
X is an anion;
R1 and R2 are independently H, a straight chain or branched alkyl group, a straight chain or branched alkene group, an electron withdrawing group, or an electron donating group and may be present or absent;
and
Y is a linking or spacer group.

2. The compound of claim 1, wherein X is selected from the group consisting of fluoride, chloride, bromide, acetate, formate, bromate, pyruvate, nitrate, isocitrate, cis-aconitate, trans-aconitate, selenium oxoanion, maleate, malonate, phosphate, citrate, sulfate, oxalate, uric acid and choline.

3. The compound of claim 1, wherein R1 is methyl, ethyl, fluoro, nitro or methoxyl.

4. The compound of claim 1, wherein R2 is methyl, ethyl, fluoro, nitro or methoxyl.

5. The compound of claim 1, wherein Y is an atom or molecule capable of forming at least two covalent bonds, a first of which is with a nitrogen moiety of Formula I and a second of which is with a phenyl moiety of Formula I.

6. The compound of claim 5, wherein Y is selected form the group consisting of CH₂, C₂H₄, COCH₂, CONH, CH₂COCH₂, CH₂CONH, CH₂COOCH₂ and CH₂CONHCH₂.

7. The compound of claim 1, wherein the compound is as shown in Formula II:

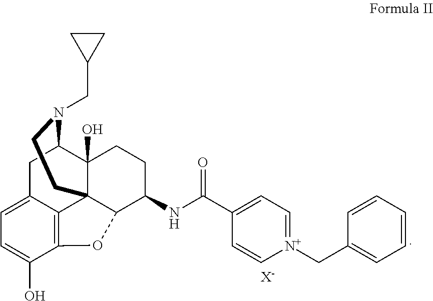

8. A method for treating opioid induced constipation in a subject in need thereof, comprising
administering to the subject a therapeutically effective amount of a compound of Formula I 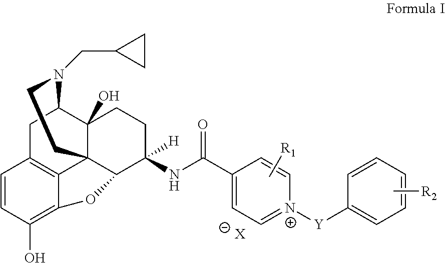
where
X is an anion;
R1 and R2 are independently H, a straight chain or branched alkyl group, a straight chain or branched alkene group, an electron withdrawing group, or an electron donating group and may be present or absent;
and
Y is a linking or spacer group.

9. The method of claim 8, wherein the compound is as shown in Formula II:

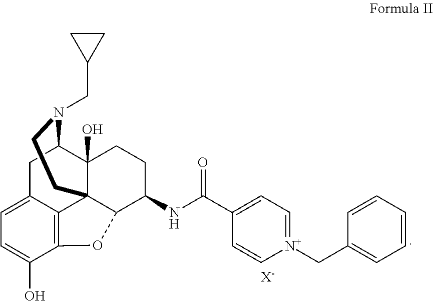

10. A method for treating irritable bowel syndrome (IBS) in a subject in need thereof, comprising
administering to the subject a therapeutically effective amount of a compound of Formula I 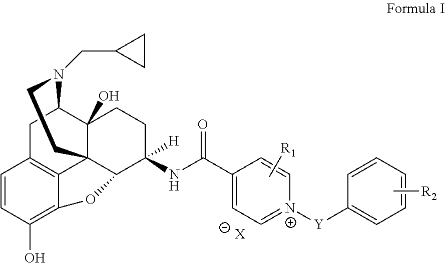
where
X is an anion;
R1 and R2 are independently H, a straight chain or branched alkyl group, a straight chain or branched alkene group, an electron withdrawing group, or an electron donating group and may be present or absent;
and
Y is a linking or spacer group.

11. The method of claim 10, wherein the compound is as shown in Formula II: [chemical structure]
